(19) United States
(12) Patent Application Publication
(10) Pub. No.: US 2004/0128161 A1
(43) Pub. Date: Jul. 1, 2004

(54) System and method for ad hoc communications with an implantable medical device

(76) Inventors: Scott T. Mazar, Inver Grove Heights, MN (US); Yatheendhar D. Manicka, Woodbury, MN (US)

Correspondence Address: MERCHANT & GOULD PC, P.O. BOX 2903, MINNEAPOLIS, MN, 55402-0903, US

(21) Appl. No.: 10/330,489

(22) Filed: Dec. 27, 2002

(51) Int. Cl. G06F 17/60; A61B 5/00

(52) U.S. Cl. 705/2; 600/300

(57) ABSTRACT

A system and method for communicating with an IMD implanted within a patient via an advanced patient management system is provided. A determination is made as to whether the patient should be notified prior to communicating with the IMD. If the patient is to be notified prior to communicating with the IMD, one or more actions to be taken may be communicated to the patient. Once the patient has been instructed to take the necessary actions and the actions have been completed, or if the patient need not be notified, a communications link between the patient management system and the IMD may be established. Once the communications link has been established, device parameters and other data may be retrieved from the IMD. Additionally, the device parameters may be modified to suit the patient's current medical condition. When the communications session has been completed, the communications link between the patient management system and the IMD is disconnected. The patient may then be notified that the session has completed and instructed to resume their normal activities.

### TECHNICAL FIELD

The present system relates generally to disease management systems, and particularly, but not by way of limitation, to methods and systems for remotely communicating in real-time with an implantable medical device.

### BACKGROUND OF THE INVENTION

Management of patients with chronic disease consumes a significant proportion of the total health care expenditure in the United States. Many of these diseases are widely prevalent and have significant annual incidences as well. Heart failure prevalence alone is estimated at over 5.5 million patients in 2000 with incidence rates of over half a million additional patients annually, resulting in a total health care burden in excess of $20 billion. Heart failure, like many other chronic diseases such as Asthma, Chronic Obstructive Pulmonary Disease (“COPD”), Chronic Pain, and Epilepsy is event driven, where acute de-compensations result in hospitalization. In addition to causing considerable physical and emotional trauma to the patient and family, event driven hospitalizations consume a majority of the total health care expenditure allocated to the treatment of heart failure.

Most acute de-compensations result in hospitalization and treatment after the event has occurred. However, most heart failure patients exhibit prior non-traumatic symptoms, such as steady weight gain, in the weeks or days prior to the de-compensation. If the attending physician is made aware of these symptoms, it is possible to intervene before the event at substantially less cost to the patient and the health care system. Intervention is usually in the form of a re-titration of the patient's drug cocktail, reinforcement of the patient's compliance with the prescribed drug regimen, or acute changes to the patient's diet and exercise regimens. Such intervention is usually effective in preventing the de-compensation episode and thus avoiding hospitalization.

In order to provide early detection of symptoms that may signal an increased likelihood of a traumatic medical event, patients may receive implantable medical devices (“IMDs”) that have the ability to measure various body characteristics. For instance, IMDs are currently available that provide direct measurement of electrical cardiac activity, physical motion, temperature, and other clinical parameters. Patients that have experienced traumatic medical events or that are at high risk of experiencing such events may receive IMDs that can also provide therapy. For instance, patients with chronic heart disease can receive implantable cardiac devices such as pacemakers, implantable cardioverter defibrillators, and HF cardiac resynchronization therapy devices.

When a physician or clinician needs to take measurements or make changes to an implanted IMD, the patient typically needs to visit a doctor's office or similar facility. Scheduling an appointment and traveling to and from the appointment may be time-consuming and burdensome for a patient. Thus, there is a need for a method and system for communicating with an implantable medical device that can communicate with the device in real-time so that device measurements and device adjustments may be practiced remotely by a clinician or physician.

It is with respect to these considerations and others that the present invention is made.

### SUMMARY OF THE INVENTION

The present invention addresses these problems and others by providing a method and system for real-time ad hoc communication with an implantable medical device. The method and systems provided herein allow physicians or others to monitor a patient's health and/or make adjustments to an implanted IMD remotely using a patient management system. A lab clinician or a physician at a remote site may use the patient management system to communicate with the IMD in real-time and on an as-needed basis. Data may be retrieved from the IMD and device parameters within the IMD may be adjusted with or without the participation or knowledge of the patient. Because the remote clinician or physician may also use the disease management system to adjust parameters or settings on the IMD in real-time, the patient does not need to travel to a clinic or a physician's office to have the adjustments made.

According to one aspect of the invention, a method is provided for communicating with an IMD implanted within a patient via an advanced patient management system. The IMD may communicate with a repeater device through an inductive or short range wireless communications link. The repeater device may communicate with the patient management system through a wired or wireless connection to the patient management system. Alternatively, the IMD may be configured for long range wireless communication with the patient management system, such as through a cell phone or wireless pager network.

In order to establish communication between the IMD and the patient management system, a determination is first made as to whether the patient should be notified prior to communicating with the IMD. The patient may be notified if only a single communications channel exists for voice communication with the patient and for data communication with a repeater device used with the IMD. For instance, if the patient only has a single phone line that is utilized for voice communication and by the repeater device. The patient may also be notified if any changes are to be made to the IMD that may effect the health or well being of the patient. If the communications session with the IMD is only to retrieve data from the IMD or for other benign purposes, the patient may not be notified. The patient may also be notified if the participation of the patient is necessary to establish communication with the IMD, such as when an inductive communications link must be established between the IMD and the repeater device.

If the patient is to be notified prior to communicating with the IMD, one or more actions to be taken may be communicated to the patient. For instance, the patient may be instructed to take special precautions prior to the start of the communications session, such as lying down. The patient may also be instructed to locate themselves proximate to a repeater device or other device necessary to establish communication with the IMD. The patient may also be instructed to configure. the repeater device for communication with the patient management system. This may also include releasing a communications link, such as a phone line, so that a data connection may be established between the repeater device and the patient management system.

Once the patient has been instructed to take the necessary actions and the actions have been completed, or if the patient need not be notified, a communications link between the patient management system and the IMD may be established. The communications link is established by a physician or technician at the patient management system. The communications link may be established by creating a telephone or network connection to the repeater device or by communicating directly with an IMD equipped with long-range wireless capabilities. Once the communications link has been established, device parameters and other data may be retrieved from the IMD. Additionally, the device parameters may be modified to suit the patient's current medical condition.

When the communications session has been completed, the communications link between the patient management system and the IMD is disconnected. The patient may then be notified that the session has completed and instructed to resume their normal activities. If the patient was not notified prior to establishing the communications link, the patient would not receive a notification following completion of the communications session.

Aspects of the invention also provide a system, apparatus, and computer-readable medium for providing real-time ad hoc communication between an IMD and a patient management system. These and various other features as well as advantages, which characterize the present invention, will be apparent from a reading of the following detailed description and a review of the associated drawings.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates an advanced patient management system utilized in one embodiment of the present invention;

FIG. 2 illustrates a computer system utilized in various embodiments of the present invention;

FIG. 3 illustrates an example interrogator/transceiver unit provided by one embodiment of the present invention;

FIG. 4 shows a communication system utilized in one embodiment of the present invention;

FIG. 5 illustrates a system for providing real-time ad hoc communications with an implantable medical device;

FIG. 6 illustrates another system for providing real-time ad hoc communications with an implantable medical device; and

FIG. 7 illustrates a method for performing ad hoc communication with an implantable medical device.

### DETAILED DESCRIPTION

In the following detailed description, reference is made to the accompanying drawings which form a part hereof, and in which is shown by way of illustration specific embodiments or examples. These embodiments may be combined, other embodiments may be utilized, and structural, logical, and electrical changes may be made without departing from the spirit and scope of the present invention. The following detailed description is, therefore, not to be taken in a limiting sense, and the scope of the present invention is defined by the appended claims and their equivalents.

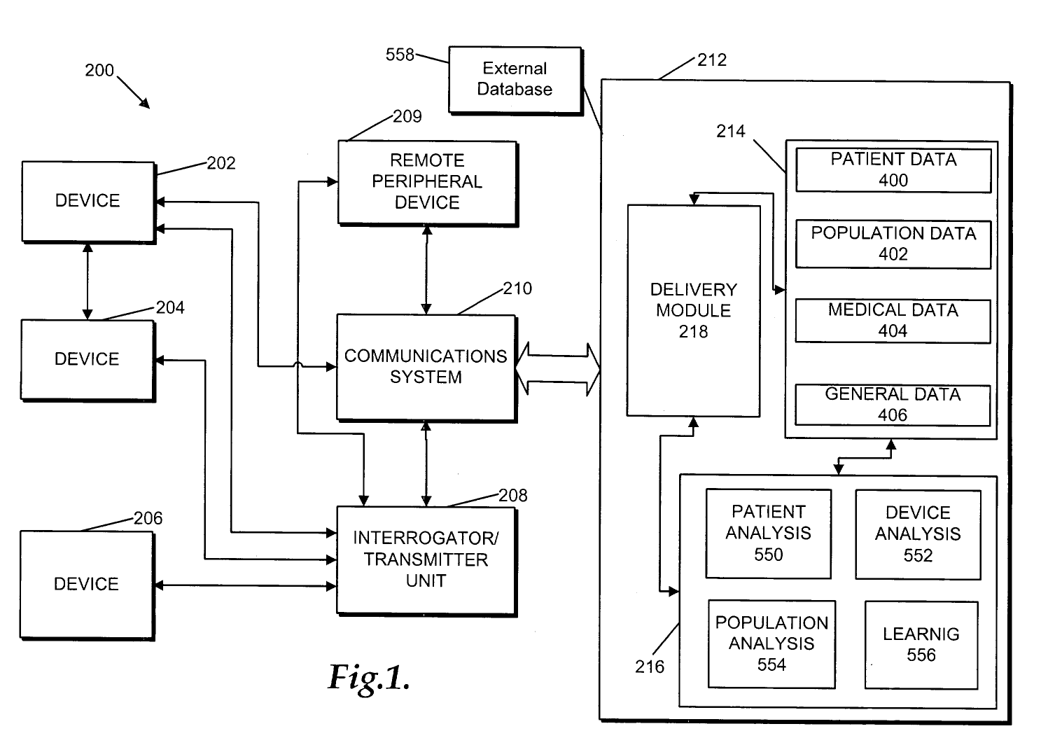

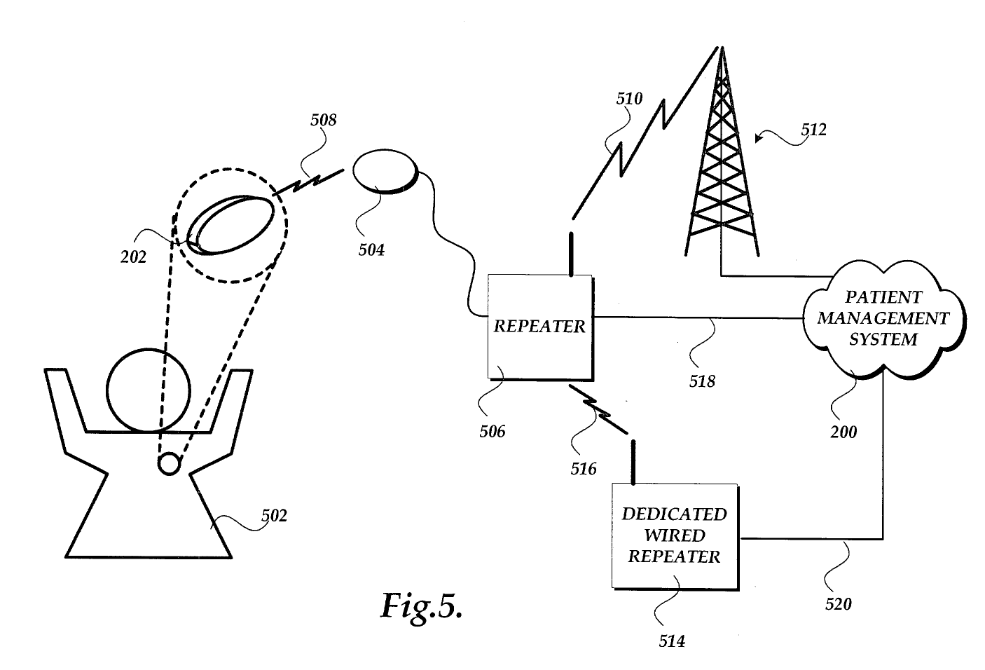

The apparatus and methods described herein are described in the context of a patient management system that provides patient management and device management. As used herein, the phrase “patient management” refers to the process of creating and collecting patient specific information, storing and collating the information, and generating actionable recommendations to enable the predictive management of patients with chronic disease. As used herein, the phrase “device management’ refers to the process of leveraging a remote communications infrastructure to provide automatic device follow-ups to collect data, provide therapy, and to determine if remote devices are functioning properly. It should be appreciated that although the embodiments of the invention are described in the context of a patient management system, the embodiments of the invention may be utilized within other operating environments. Additional details regarding the patient management system that provides one operating environment for the embodiments of the invention are provided below with respect to FIGS. 1-4. Additional details regarding the methods and systems provided herein for ad hoc communication with an implantable medical device are provided below with respect to FIGS. 5-7.

As discussed briefly above, embodiments of the present invention are described with respect to an advanced patient management system configured to collect patient specific information, store and collate the information, and generate actionable recommendations to enable the predictive management of patients. The advanced patient management system is also configured to leverage a remote communications infrastructure to provide automatic device follow-ups to collect data, provide therapy, and to determine if remote devices are functioning properly. The term “patient” is used herein to mean any individual from whom information is collected. The term “caregiver” is used herein to mean any provider of services, such as health care providers including, but not limited to, nurses, doctors, and other health care provider staff.

FIG. 1 illustrates an example advanced patient management system 200 made in accordance with the present invention. The advanced patient management system 200 can generally include the following components: one or more devices 202, 204, and 206, one or more interrogator/transceiver units 208, a communications system 210, one or more remote peripheral devices 209, and a host 212.

Each component of the advanced patient management system 200 can communicate using the communications system 210. Some components may also communicate directly with one another. For example, devices 202 and 204 may be configured to communicate directly with one another. The various components of the example advanced patient management system 200 illustrated herein are described below.

Devices 202, 204, and 206 can be implantable devices or external devices that may provide one or more of the following functions with respect to a patient: (1) sensing, (2) data analysis, and (3) therapy. For example, in one embodiment, devices 202, 204, and 206 can be implanted or external devices used to measure a variety of physiological, subjective, and environmental conditions of a patient using electrical, mechanical, and/or chemical means. The devices 202, 204, and 206 can be configured to automatically gather data or can require manual intervention by the patient. The devices 202, 204, and 206 can be configured to store data related to the physiological and/or subjective measurements and/or transmit the data to the communications system 210 using a variety of methods, described in detail below. Although three devices 202, 204, and 206 are illustrated in the example embodiment shown, more or fewer devices may be used for a given patient.

The devices 202, 204, and 206 can be configured to analyze the measured data and act upon the analyzed data. For example, the devices 202, 204, and 206 may be configured to modify therapy or provide alarm indications based on the analysis of the data. In one embodiment, devices 202, 204, and 206 may also provide therapy. Therapy can be provided automatically or in response to an external communication. Devices 202, 204, and 206 can be programmable in that the characteristics of their sensing (e.g., duration and interval), therapy, or communication can be altered via communication between the devices 202, 204, and 206 and other components of the advanced patient management system 200. Devices 202, 204, and 206 can also perform self-checks or be interrogated by the communications system 210 to verify that the devices are functioning properly. Examples of different embodiments of the devices 202, 204, and 206 are provided below.

Devices implanted within the body have the ability to sense and communicate as well as to provide therapy. Implantable devices can provide direct measurement of characteristics of the body, including, without limitation, electrical cardiac activity (e.g., a pacemaker, cardiac resynchronization management device, defibrillator, etc.), physical motion, temperature, heart rate, activity, blood pressure, breathing patterns, ejection fractions, blood viscosity, blood chemistry, blood glucose levels, and other patient-specific clinical physiological parameters, while minimizing the need for patient compliance.

A heart rhythm sensor, typically found in a pacemaker or defibrillator, is one example of implantable device. In the heart, an electrical wave activates the heart muscle just prior to contraction. As is known in the art, electrical circuits and lead-wires transduce the heart's activation event and reject other, non-essential electrical events. By measuring the time interval between activation events, the heart rhythm can be determined. A transthoracic impedance sensor is another example of an implantable device. During the respiratory cycle, large volumes of air pass into and out of the body. The electrical resistance of the thorax changes markedly as a result of large differences in conductivity of air and body tissues. The thoracic resistance can be measured during respiration and converted into a measurable electrical signal (i.e., impedance) so that breathing rate and profile can be approximated. Implantable devices can also sense chemical conditions, such as glucose levels, blood oxygen levels, etc. Further, the advanced patient management system 200 may utilize other implantable devices as well that provide physiological measurements of the patient, such as drug pumps, neurological devices (e.g., stimulators), oxygen sensors, etc.

Derived measurements can also be determined from the implantable devices. For example, a sleep sensor can rely on measurements taken by an implanted accelerometer that measures body activity levels. The sleep sensor can estimate sleeping patterns based on the measured activity levels. Other derived measurements can include a functional capacity indicator, autonomic tone indicator, sleep quality indicator, cough indicator, anxiety indicator, and cardiovascular wellness indicator for calculating a quality of life indicator for quantifying a patient's overall health and well-being.

Devices 202, 204, and 206 can also be external devices, or devices that are not implanted in the human body, that may be used to measure physiological data. Such devices may include a multitude of devices to measure data relating to the human body, including temperature (e.g., a thermometer), blood pressure (e.g., a sphygmomanometer), blood characteristics (e.g., glucose levels), body weight, physical strength, mental acuity, diet, heart characteristics, and relative geographic position (e.g., a Global Positioning System (“GPS”)).

Devices 202, 204, and 206 can also be environmental sensors. The devices can be placed in a variety of geographic locations (in close proximity to patient or distributed throughout a population) and can record non-patient specific characteristics such as, for example, temperature, air quality, humidity, carbon monoxide level, oxygen level, barometric pressure, light intensity, and sound.

One or more of the devices 202, 204, and 206 (for example, device 206) may be external devices that measure subjective or perceptive data from the patient. Subjective data is information related to a patient's feelings, perceptions, and/or opinions, as opposed to objective physiological data. For example, the “subjective” devices can measure patient responses to inquiries such as “How do you feel?” and “How is your pain?” and “Does this taste good?”. The device can prompt the patient and record subjective data from the patient using visual and/or audible cues. For example, the patient can press coded response buttons or type an appropriate response on a keypad. Alternatively, subjective data may be collected by allowing the patient to speak into a microphone and using speech recognition software to process the subjective data.

In one example embodiment, the subjective device presents the patient with a relatively small number of responses to each question posed to the patient. For example, the responses available to the patient may include three faces representing feelings of happiness, nominalness, and sadness. Averaged over time, a trend of a patient's well being may emerge with a finer resolution than the quanta of the three responses. The subjective data can be collected from the patient at set times, or, alternatively, can be collected whenever the patient feels like providing subjective data. The subjective data can also be collected substantially contemporaneously with physiological data to provide greater insight into overall patient wellness.

The device 206 can be any device that accepts input from a patient or other concerned individual and/or provides information in a format that is recognizable to the patient. Device 206 can typically include a keypad, mouse, display, handheld device, interactive TV, a cellular telephone or other radio frequency (“RF”) communications device, cordless phone, corded phone, speaker, microphone, email message, and physical stimulus such as an electric shock or change in temperature or light intensity.

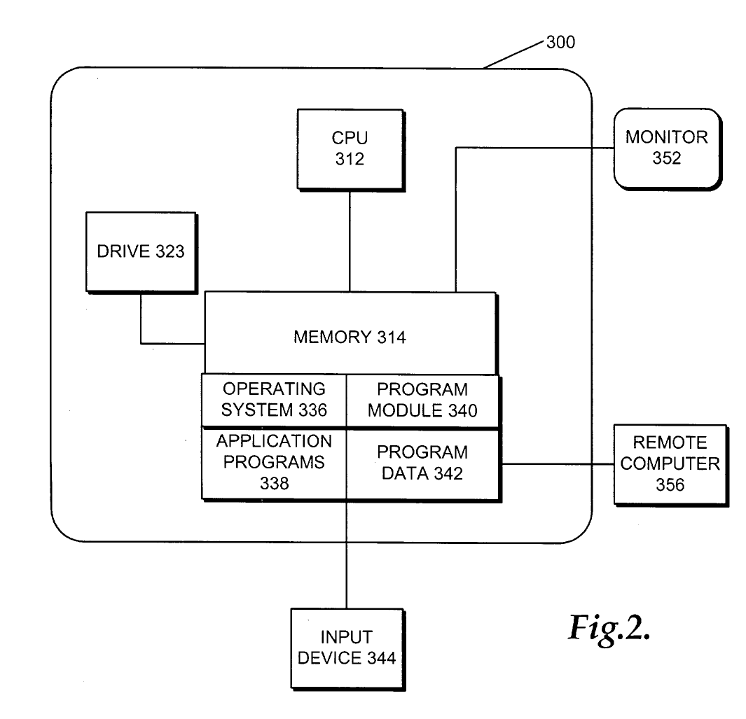

In one example embodiment, the device 206 includes or is part of a computer system 300, as illustrated in FIG. 2. The computer system 300 can include a central processor unit 312 and a system memory 314. The computer system 300 further includes one or more drives 323 for reading data from and writing data to, as well as an input device 344 such as a keyboard or mouse and a monitor 352 or other type of display device.

A number of program modules may be stored on the drive 323, including an operating system 336, one or more application programs 338, other program modules 340, and program data 342. The computer system 300 may operate in a networked environment using logical connections to one or more remote computers or computer systems 356. Computer system 300 may also comprise a hand-held computer such as a personal digital assistant (“PDA”) computer.

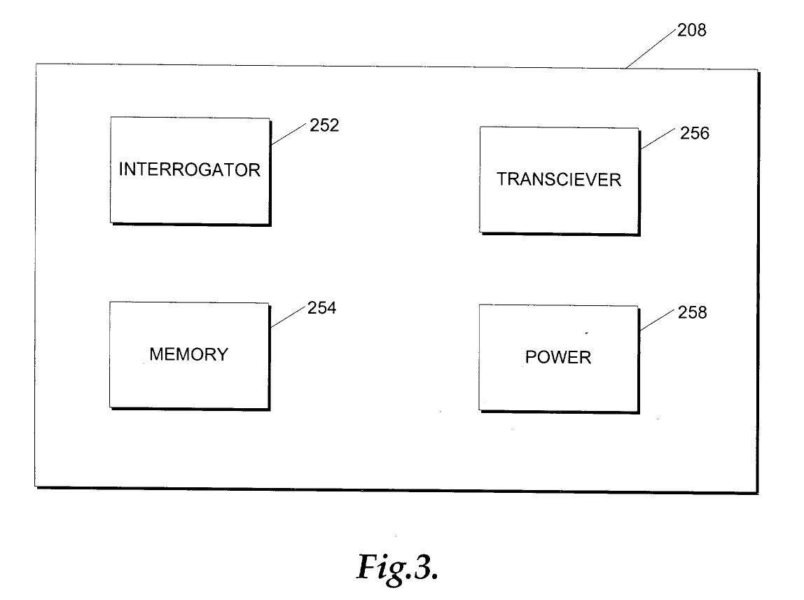

Referring now to FIG. 3, the advanced patient management system 200 may include one or more interrogator/transceiver units (“ITUs”), such as ITU 208. The ITU 208 includes an interrogator module 252 for receiving data from a device such as devices 202, 204, and 206, a memory module 254 for storing data, a transceiver module 256 for sending data both to the devices 202, 204, and 206 as well as other components of the advanced patient management system 200. The ITU 208 also includes a power module 258 that provides power.

The ITU 208 may perform one or more of the following functions: (1) data storage; (2) data analysis; (3) data forwarding; (4) patient interaction; and (5) patient feedback. For example, the ITU 208 may facilitate communications between the devices 202, 204, and 206 and the communications system 210. The ITU 208 can, periodically or in real-time, interrogate and download into memory clinically relevant patient data from the devices 202, 204, and/or 206. This data can include, in the cardiac sensor context, for example, P and R-Wave measurements, pacing, shocking events, lead impedances, pacing thresholds, battery voltage, capacitor charge times, ATR episodes with electrograms, tachycardia episodes with electrograms, histogram information, and any other clinical information necessary to ensure patient health and proper device function. The data may be sent to the ITU 208 by the devices 202, 204, and 206 in real-time or periodically uploaded out of buffers on the devices.

The ITU 208 may also allow for patient interaction. For example, the ITU 208 may include a patient interface and allow the patient to input subjective data. In addition, the ITU 208 may provide feedback to the patient based on the data that has been analyzed or based on information communicated by the communications system 210.

In another embodiment, the ITU 208 can include a telemetry link from the implanted device to a network that forms the basis of a wireless LAN in the patient's home. The device can systematically download information from the devices 202, 204, and 206 while the patient is sleeping, for example. The data can be transmitted by landline or wirelessly to the communications system 210 or directly to the host 212. In addition, in one embodiment the ITU 208 can function in a hybrid form, utilizing wireless communication when available and defaulting to landline communication when the wireless communication becomes unavailable.

Some devices, such as legacy implanted cardiac rhythm management (“CRM”) devices, communicate via an internal telemetry transceiver that communicates with an external programmer. The communication range of such devices is typically a few inches. Communications system 210 may include a special purpose “ITU” that communicates with an implanted legacy device, on one hand, and communicates with the wireless Internet on the other. Patients with legacy devices are provided with these ITUs and are instructed to use them periodically (e.g., monthly).

The ITU 208 may be in the form of a small device that is placed in an inconspicuous place within the patient's residence. Alternatively, the ITU may be implemented as part of a commonly used appliance in the patient's residence. For example, the ITU may be integrated with an alarm clock that is positioned near the patient's bed. In another embodiment, the ITU may be implemented as part of the patient's personal computer system. Other embodiments are also possible.

In another embodiment, the ITU 208 may comprise a hand-held device such as a PDA, cellular telephone, or other similar device that is in wireless communication with the devices 202, 204, and 206. The hand-held device may upload the data to the communications system 210 wirelessly. Alternatively, the hand-held device may periodically be placed in a cradle or other similar device that is configured to transmit the data to the communications system 210.

The ITU 208 can also perform analysis on the data and provide immediate feedback, as well as perform a variety of self-diagnostic tests to verify that it is functioning properly and that communication with the communications system 210 has not be compromised. For example, the ITU 208 can perform a diagnostic loop-back test, which involves sending a request through the communications system 210 to the host 212. The host 212 can then reply with a response back through the communications system 210 to the ITU 208. If a specific duration elapses before the ITU 208 receives the response, or if the ITU 208 receives an unexpected response, the ITU 208 can provide indications that the system is not functioning properly. For example, if wireless communications between the ITU 208 and the communications system 210 have been interrupted, and the ITU 208 performs a self-diagnostic test that fails, the ITU 208 may alert data management service personnel so that corrective action may be taken. Alternatively, the ITU 208 can sound a visual and/or audible alarm to alert the patient that communication has been interrupted. In another embodiment, the ITU 208 can automatically fail-back to a landline system to communicate with the communications system 210.

In other embodiments of the advanced patient management system 200, the ITU 208 can be eliminated completely, and the devices 202, 204, and 206 can communicate directly with the communications system 210 and/or host 212. For example, device 202 may include a miniature cellular phone capable of wirelessly uploading clinical data from the device on a periodic basis. This is particularly advantageous for devices that are mobile (e.g., an implanted device in a patient that is traveling). The device 202 can incorporate wireless telecommunications such as cellular, BLUETOOTH, or IEEE 802.11B to communicate with the communications system 210.

To conserve the energy of the devices 202, 204, and 206, particularly when the devices (e.g., device 202) are configured to communicate directly with the communications system 210 without using an ITU, in one example embodiment the devices are configured to communicate during a given duty cycle. For example, the device 202 can be configured to communicate with the communications system 210 at given intervals, such as once a week. The device 202 can record data for the time period (e.g., a week) and transmit the data to the communications system 210 during the portion of the cycle that transmission is active and then conserve energy for the rest of the cycle. In another example, the device 202 conserves energy and only communicates with the communications system 210 when an “interesting” event, such as a heart arrhythmia, has occurred. In this manner, device 202 can communicate directly with the communications system 210 and/or host 212 without using the ITU 208, while conserving the energy of the device by communicating only during a given duty cycle.

If multiple devices, such as devices 202, 204, and 206, are provided for a given patient, each device may include its own means for communicating with the ITU 208 or communications system 210. Alternatively, a single telemetry system may be implemented as part of one of the devices, or separate from the devices, and each device 202, 204, and 206 can use this single telemetry system to communication with the ITU 208 or the communications system 210.

In yet another embodiment, the devices 202, 204, and 206 include wires or leads extending from devices 202, 204, and 206 to an area external of the patient to provide a direct physical connection. The external leads can be connected, for example, to the ITU 208 or a similar device to provide communications between the devices 202, 204, and 206 and the other components of the advanced patient management system 200.

The advanced patient management system 200 can also involve a hybrid use of the ITU 208. For example, the a device such as devices 202, 204, and 206 can intelligently communicate via short-range telemetry with the ITU when the patient is located within the patient's home and communicate directly with the communications system 210 or host 212 when the patient is traveling. This may be advantageous, for example, to conserve battery power when the devices are located near an ITU.

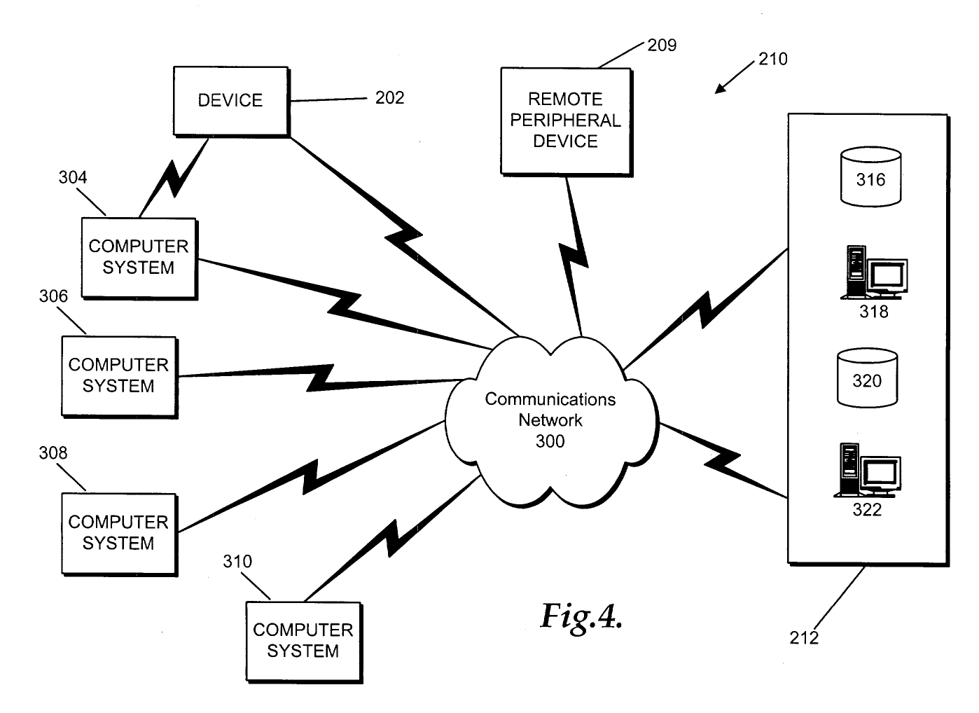

Communications system 210 provides for communications between and among the various components of the advanced patient management system 200, such as the devices 202, 204, and 206, host 212, and remote peripheral devices 209. FIG. 4 illustrates communications system 210 according one embodiment of the present invention. The communications system 210 includes a plurality of computer systems 304, 306, 308, and 310, as well as device 202, host 212, and remote peripheral device 109, connect to one another by the communications network 300. The communications network 300 may be, for example, a local area network (“LAN”), wide area network (WAN), or the Internet. Communications among the various components, as described more fully below, may be implemented using wired or wireless technologies.

In the example embodiment illustrated, the host 212 includes server computers 318 and 322 that communicate with computers 304, 306, 308, and 310 using a variety of communications protocols, described more fully below. The server computers 318 and 322 may store information in databases 316 and 320. This information may also be stored in a distributed manner across one or more additional servers.

As shown in FIG. 4, a variety of communication methods and protocols may be used to facilitate communication between devices 202, 204, and 206, ITU 208, communications system 210, host 212, and remote peripheral device 109. For example, wired and wireless communications may be used. Wired communication methods may include, for example and without limitation, traditional copper-line communications such as DSL, broadband technologies such as ISDN and cable modems, and fiber optics, while wireless communications may include cellular, satellite, radio frequency (“RF”), Infrared, etc.

For any given communication method, a multitude of standard and/or proprietary communication protocols may be used. For example and without limitation, wireless (e.g., radio frequency pulse coding, spread spectrum, direct sequence, time-hopping, frequency hopping, etc.) and other communication protocols (e.g., SMTP, FTP, TCP/IP) may be used. Other proprietary methods and protocols may also be used. Further, a combination of two or more of the communication methods and protocols may also be used.

The various communications between the components of the advanced patient management system 200 may be made securely using several different techniques. For example, encryption and/or tunneling techniques may be used to protect data transmissions. Alternatively, a priority data exchange format and interface that are kept confidential may also be used. Authentication can be implemented using, for example, digital signatures based on a known key structure (e.g., PGP or RSA). Other physical security and authentication measures may also be used, such as security cards and biometric security apparatuses (e.g., retina scans, iris scans, fingerprint scans, veinprint scans, voice, facial geometry recognition, etc.). Conventional security methods such as firewalls may be used to protect information residing on one or more of the storage media of the advanced patient management system 200. Encryption, authentication and verification techniques may also be used to detect and correct data transmission errors.

Communications among the various components of the advanced patient management system 200 may be enhanced using compression techniques to allow large amounts of data to be transmitted efficiently. For example, the devices 202, 204, and 206 may compress the information recorded from the patient prior to transmitting the information to the ITU 208 or directly to the communications system 210. The communication methods and protocols can facilitate periodic and/or real-time delivery of data.

The host 212 may include a database module 214, an analysis module 216, and a delivery module 218 (shown in FIG. 1). The host 212 preferably includes enough processing power to analyze and process large amounts of data collected from each patient, as well as to process statistics and perform analysis for large populations. For example, the host 212 may include a mainframe computer or multi-processor workstation. The host 220 may also include one or more commercial personal computer systems containing sufficient computing power and memory. The host 220 may include storage medium (e.g. hard disks, optical data storage devices, etc.) sufficient to store the massive amounts of high-resolution data that are collected from the patients and analyzed.

The host 212 may also include identification and contact information (e.g., IP addresses and/or telephone numbers) for the various devices communicating with it, such as ITU 208 and peripheral device 209. For example, each ITU 208 may be assigned a hard-coded or static identifier (e.g., IP address, telephone number, etc.), which would allow the host 212 to identify which patient's information the host 212 is receiving at a given instant. Alternatively, each device 202, 204, and 206 may be assigned a unique identification number, or a unique patient identification number may be transmitted with each transmission of patient data.

When a device is first activated, several methods may be used to associate data received by the advanced patient management system 200 with a given patient. For example, each device may include a unique identification number and a registration form that may be filled out by the patient, caregiver, or field representative. The registration form can be used to collect the necessary information to associate collected data with the patient. Alternatively, the user could logon to a web site to allow for the registration information to be collected. Another possible method involves including a barcode on each device that can be scanned prior to or in conjunction with initial measurements to provide information to associate the recorded data with the given patient.

Referring again to FIG. 1, the database module 214 can include a patient database 400, a population database 402, a medical database 404, and a general database 406, all described further below. The patient database 400 includes patient specific data, including data acquired by the devices 202, 204, and 206. The patient database 400 can also include a patient's medical records. The patient database 400 can include historical information regarding the devices 202, 204, and 206. For example, if device 202 is an ICD, the patient database 400 can record the following device information: P and R measurements, pacing frequency, pacing thresholds, shocking events, recharge time, lead impedance, battery voltage/remaining life, ATR episode and EGMs, histogram information, and other device information. The information stored in the database 400 can be recorded at various times depending on the patient requirements or device requirements. For example, the database 400 can be updated at periodic intervals that coincide with the patient downloading data from the device. Alternatively, data in the database 400 can be updated in real time. Typically, the sampling frequency will depend on the health condition being monitored and the co-morbidities

The population database 402 includes non-patient specific data, such as data relating to other patients and population trends. The population database 402 also records epidemic-class device statistics and patient statistics. The population database 402 also includes data relating to staffing by health care providers, environmental data, pharmaceuticals, etc.

The medical database 404 includes clinical data relating to the treatment of diseases. For example, the medical database 404 can include historical trend data for multiple patients in the form of a record of progression of their disease(s) along with markers of key events.

The general database 406 includes non-medical data of interest to the patient. This can include information relating to news, finances, shopping, technology, entertainment, and sports. The general database 406 can be customized to provide general information of specific interest to the patient. For example, stock information can be presented along with the latest health information as detected from the devices 202, 204, and 206.

In another embodiment, information may also be provided from an external source such as external database 558. For example, the external database may include external medical records maintained by a third party, such as drug prescription records maintained by a pharmacy providing information related to what types of drugs have been prescribed for a patient. The analysis module 216 includes a patient analysis module 550, device analysis module 552, population analysis module 554, and learning module 556.

The patient analysis module 550 may utilize information collected by the advanced patient management system 200, as well as information for other relevant sources, to analyze data related to a patient and provide timely and predictive assessments of the patient's well-being. In performing this analysis, the patient device module 550 may utilize data collected from a variety of sources, include patient specific physiological and subjective data collected by the advanced patient management system 200, medical and historical records (e.g., lab test results, histories of illnesses, etc., drugs currently and previously administered, etc.), as well as information related to population trends provided from sources external to the advanced patient management system 200.

For example, in one embodiment, the patient analysis module 550 may make a predictive diagnosis of an oncoming event based on information stored in the database module 214. For example, the data continuously gathered from a device of a given patient at a heightened risk for a chronic disease event (such as de-compensations in heart failure) can be analyzed. Based on this analysis, therapy, typically device-based or pharmaceutical, can then be applied to the patient.

In another example embodiment, the patient analysis module 550 may provide a diagnosis of patient health status and predicted trend based on present and recent historical data collected from a device as interpreted by a system of expert knowledge derived from working practices within clinics. For example, the patient analysis module 550 may perform probabilistic calculations using currently collected information combined with regularly collected historical information to predict patient health degradation.

In another example embodiment, the patient analysis module 550 may conduct pre-evaluation of the incoming data stream combined with patient historical information and information from patients with similar disease states. The pre-evaluation system is based on data derived from working clinical practices and the records of outcomes. The derived data can be processed into a neural network or equivalent system to reflect the clinical practice. Further, the patient analysis module 550 may also provide means for periodic processing of present and historical data to yield a multidimensional health state indication along with disease trend prediction, next phase of disease progression co-morbidities, and inferences about what other possible diseases may be involved. The patient analysis module 550 may also integrate data collected from internal and external devices with subjective data to optimize management of overall patient health.

The device analysis module 552 analyzes data from the devices 202, 204, and 206 and ITU 208 to predict and determine device failures. For example, if an implanted device 202 fails to communicate at an expected time, device analysis module 552 determines the source of the failure and takes action to restore the performance of the device 202.

The device analysis module 552 may also perform additional deterministic and probabilistic calculations. For example, the device analysis module 552 may gather data related to charge levels within a given device, such as an ICD, and provide analysis and alerting functions based on this information if, for example, the charge level reaches a point at which replacement of the device and/or battery is necessary. Similarly, early degradation or imminent failure of implanted devices can be identified and proactively addressed, or at-risk devices can be closely monitored.

The population analysis module 554 uses the data collected in the database module 214 to manage the health of a population. For example, a clinic managing cardiac patients can access the advanced patient management system 200 and thereby obtain device-supplied advance information to predict and optimize resource allocation both as to immediate care and as a predictive metric for future need of practicing specialists. As another example, the spread of disease in remote populations can be localized and quarantined rapidly before further spread.

In one embodiment, population analysis module 554 trends the patient population therapy and management as recorded by the devices and directs health care resources to best satisfy the needs of the population. The resources can include people, facilities, supplies, and/or pharmaceuticals. In other embodiments, the population analysis module can detect epidemics and other events that affect large population groups. The population analysis module 554 can issue alerts that can initiate a population quarantine, redirect resources to balance size of staffing with number of presenting population, and predict future need of qualified specialists.

The population analysis module 554 may utilize a variety of characteristics to identify like-situated patients, such as, for example, sex, age, genetic makeup, etc. The population analysis module 554 may develop large amounts of data related to a given population based on the information collected by the advanced patient management system 200. In addition, the population analysis module 554 may integrate information from a variety of other sources. For example, the population analysis module 554 may utilized data from public domain databases (e.g. National Institute of Health), public and governmental and health agency databases, private insurance companies, medical societies (e.g. American Heart Association), and genomic records (e.g., DNA sequences).

In one embodiment of the invention, the host 212 may be used as a “data clearinghouse,” to gather and integrate data collected from the devices 202, 204, and 206, as well as data from sources outside the advanced patient management system 200. The integrated data can be shared with other interested entities, subject to privacy restrictions, thereby increasing the quality and integration of data available.

The learning module 556 analyzes the data provided from the various information sources, including the data collected by the advanced patient management system 200 and external information sources. For example, the learning module 556 analyzes historical symptoms, diagnoses, and outcomes along with time development of the diseases and co-morbidities. The learning module 556 can be implemented via a neural network (or similar) system.

The learning module 556 can be partially trained (i.e., the learning module 556 may be implemented with a given set of preset values and then learn as the advanced patient management system functions) or untrained (i.e., the learning module 556 is initiated with no preset values and must learn from scratch as the advanced patient management system functions). In other alternative embodiments, the learning module 556 may continue to learn and adjust as the advanced patient management system functions (i.e., in real time), or the learning module 556 may remain at a given level of learning and only advanced to a higher level of understanding when manually allowed to do so.

The learning module 556 may implement various algorithms and mathematical modeling such as, for example, trend and statistical analysis, data mining, pattern recognition, cluster analysis, neural networks and fuzzy logic. Learning module 556 may perform deterministic and probabilistic calculations. Deterministic calculations include algorithms for which a clear correlation is known between the data analyzed and a given outcome. For example, there may be a clear correlation between the power left in a battery of an implantable device and the amount of time left before the battery must be replaced.

A probabilistic calculation involves the correlation between data and a given outcome that is less than 100 percent certain. Probabilistic determinations require an analysis of several possible outcomes and an assignment of probabilities for those outcomes (e.g., an increase in weight of a patient may, at a 25% probability, signal an impending de-compensation event and/or indicate that other tests are needed). The learning module 556 may perform probabilistic calculations and select a given response based on less than a 100% probability. Further, as the learning module 556 “learns” for previous determinations (e.g., through a neural network configuration), the learning module 556 may become more proficient at assigning probabilities for a given data pattern, thereby being able to more confidently select a given response. As the amount of data that has been analyzed by the learning module 556 grows, the learning module 556 may become more and more accurate at assigning probabilities based on data patterns. A bifurcated analysis may be performed for diseases exhibiting similar symptoms.

In addition, patient specific clinical information can be stored and tracked for hundreds of thousands of individual patients, enabling a first-level electronic clinical analysis of the patient's clinical status and an intelligent estimate of the patient's short-term clinical prognosis. The learning module 556 may be capable of tracking and forecasting a patient's clinical status with increasing levels of sophistication by measuring a number of interacting co-morbidities, all of which may serve individually or collectively to degrade the patient's health. This will enable learning module 556, as well as caregivers, to formulate a predictive medical response to oncoming acute events in the treatment of patients with chronic diseases such as heart failure, diabetes, pain, cancer, and asthma/COPD, as well as possibly head-off acute catastrophic conditions such as MI and stroke.

In a neural network embodiment, new clinical information is presented to create new neural network coefficients that are distributed as a neural network knowledge upgrade. The learning module 556 can include a module for verifying the neural network conclusions for clinical accuracy and significance. The learning module 556 can analyze a database of test cases, appropriate outcomes and relative occurrence of misidentification of the proper outcomes. In.some embodiments, the learning module 556 can update the analysis module 216 when the analysis algorithms exceed a threshold level of acceptable misidentifications.

The delivery module 218 coordinates the delivery of feedback based on the analysis performed by the host 212. In response to the analysis module 216, delivery module 218 can manage the devices 202, 204, and 206, perform diagnostic data recovery, program the devices, and otherwise deliver information as needed.

In some embodiments, the delivery module 218 can manage a web interface that can be accessed by patients or caregivers. The information gathered by an implanted device can be periodically transmitted to a web site that is securely accessible to the caregiver and/or patient in a timely manner. In other embodiments a patient accesses detailed health information with diagnostic recommendations based upon analysis algorithms derived from leading health care institutions.

For example, the caregiver and/or patient can access the data and analysis performed on the data by accessing one or more general content providers. In one example, the patient's health information is accessed through a general portal such as MY YAHOO provided by YAHOO! INC. of Sunnyvale, California. A patient can access his or her MY YAHOO homepage and receive information regarding current health and trends derived from the information gathered from the devices 202, 204, and 206, as well as other health information gathered from other sources. The patient may also access information other than health information on the MY YAHOO website, such as weather and stock market information. Other electronic delivery methods such as email, facsimile, etc. can also be used.

In an alternative embodiment, the data collected and integrated by the advanced patient system 200, as well as any analysis performed by the system 200, can be delivered by delivery module 218 to a caregiver's hospital computer system for access by the caregiver. A standard or custom interface can facilitate communications between the advanced patient management system 200 and a legacy hospital system used by the caregiver so that the caregiver can access all relevant information using a system familiar to the caregiver.

In addition, the advanced patient management system 200 can be configured so that various components of the system (e.g., ITU 208, communications system 210, and/or host 212) provide reporting to various individuals (e.g., patient and/or caregiver). For example, different levels of reporting can be provided by (1) the ITU 208 and (2) the host 212. For example, the ITU 208 may be configured to conduct rudimentary analysis of data gathered from devices 202, 204, and 206, and provide reporting should an acute situation be identified. For example, if the ITU 208 detects that a significant heart arrhythmia is imminent or currently taking place, the ITU 208 can provide reporting in the form of an audible or visual alarm.

The host 212 can provide a more sophisticated reporting system. For example, the host 212 may provide exception-based reporting and alerts that categorize different reporting events based on importance. Some reporting events may not require caregiver intervention and therefore can be reported automatically. In other escalating situations, caregiver and/or emergency response personnel may need to become involved. For example, based on the data collected by the advanced patient management system 200, the delivery module 218 can communicate directly with the devices 202, 204, and 206, contact a pharmacy to order a specific medication for the patient, and/or contact 911 emergency response. In an alternative embodiment, the delivery module 218 and/or the patient may also establish a voice communication link between the patient and a caregiver, if warranted.

In addition to forms of reporting including visual and/or audible information, the advanced patient management system 200 can also communicate with and reconfigure one or more of the devices 202, 204, and 206. For example, if device 202 is part of a cardiac rhythm management system, the host 212 and communicate with the device 202 and reconfigure the therapy provided by the cardiac rhythm management system based on the data collected from one or more of the devices 202, 204, and 206. In another embodiment, the delivery module 218 can provide to the ITU 208 recorded data, an ideal range for the data, a conclusion based on the recorded data, and a recommended course of action. This information can be displayed on the ITU 208 for the patient to review.

The advanced patient management system 200 may also include one or more remote peripheral devices 209. The remote peripheral device 209 may include, for example and without limitation,.cellular telephones, pagers, PDA devices, facsimiles, remote computers, printers, video and/or audio devices, etc. The remote peripheral device 209 may communicate using landline or wireless technologies and may be used by the patient or caregiver to communicate with the communications system 210 and/or the host 212. For example, the remote peripheral device 209 may be used by a caregiver to receive alerts from the host 212 based on data collected from the patient and to send instructions from the caregiver to either the patient or other clinical staff. In another example, the remote peripheral device 209 may be used by the patient to receive periodic or real time updates and-alerts regarding the patient's health and well-being.

Referring now to FIG. 5, an illustrative operating environment for one actual embodiment of the present invention will be described. As shown in FIG. 5, an implantable medical device 202 is provided that may be implanted within a patient 502. The device 202 has the ability to sense and communicate and may also provide therapy. In particular, the device 202 includes a sensor which allows it to directly measure characteristics of the patient's body. This may include monitoring electrical cardiac activity, physical motion, temperature, heart rate, activity, blood pressure, breathing patterns, wedge-pressure, ejection fractions, blood viscosity, blood chemistry, blood glucose levels, or other patient specific clinical parameters without any patient compliance.

In one embodiment of the invention, interactive access to the device 202 is provided, thereby allowing physicians or other third parties to monitor the health of a patient 502 and/or make adjustments to the device 202 remotely using the patient management system 200 described above with respect to FIGS. 1-5. In various embodiments, the invention is a method or system for allowing a clinician or other health care provide to gain interactive access to a device 202 for the purposes of real-time device communication of device data and parameters and patient measurements. In addition, adjustments to the device 202 may be remotely generated in real-time.

In some embodiments of the invention, the device 202 communicates with a repeater 506 located in proximity to the patient and thus, in proximity to the device 202. The device 202 may communicate with the repeater via an inductive communications link 508. For example, a wand device 504 may be placed over the area of the patient's body where the device 202 is located, such as the chest area. The wand 504 may receive signals from the device 202 and communicate those signals to the repeater via a wired connection to the repeater 506. As will be described in greater detail below, the device 202 may also be connected to the repeater 506 via a wireless connection. For example, the device 202 may transmit RF signals that are received by the repeater 506.

In some embodiments of the invention, the repeater 506 is a dedicated, wired repeater. The repeater 506 is connected via a wired connection 518 to the remainder of the patient management system 200. The wired connection 518 to the remainder of the patient management system 200 may be a telephone line, a Digital Subscriber Line (“DSL”) connection, a cable modem connection, a Ti line, or another wired real-time connection.

In other embodiments of the invention, the repeater 506 may be a wireless repeater that uses a wireless communications link 510 and a wireless communications system 512 to communicate with the patient management system 200. The wireless communications system 512 may be a cellular system, a paging system, a PCS system or another wireless system well-known to those skilled in the art. Moreover, according to other embodiments of the invention, the repeater 506 may be a portable repeater that utilizes a short range wireless communications link 516 to communicate with a dedicated wired repeater 514. The dedicated wired repeater 514 is a stationary unit that maintains a wired connection 520 to the patient management system 200. By utilizing a portable repeater 506, the patient may gain additional mobility not otherwise available with a fixed location dedicated wired repeater 514.

In one embodiment of the invention, a lab clinician or a physician at a remote site may use the patient management system 200 to communicate with the device 202, thus receiving patient information. The remote clinician or physician may also use the patient management system 200 to adjust parameters or settings on the device in real-time. Thus, the patient does not need to travel to a clinic or physician's office to have the adjustments made.

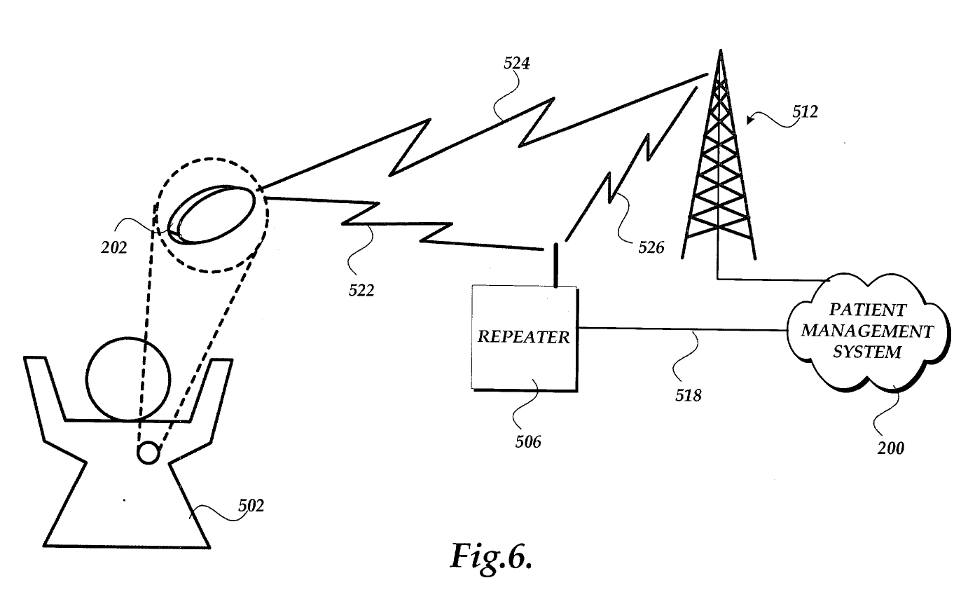

Referring now to FIG. 6, an illustrative operating environment for other embodiments of the present invention will be described. As shown in FIG. 6, the implantable medical device 202 provided herein may also include a wireless transmitter/receiver unit capable of establishing a wireless communications link 524 with a wireless communications system 512. In an actual embodiment of the present invention, the wireless communications system 512 comprises a digital wireless telephone network. In this embodiment, the implantable medical device 202 communicates directly with a cell tower to establish a communications link to the patient management system 200.

According to one embodiment, the implantable medical device 202 establishes a connection with the wireless communications system 512 in the same way that a traditional cellular telephone would establish such a connection and no repeater device is necessary. Through the wireless communications system 512, a data communications link can be established with the implantable medical device 202 for communicating with the patient management system 200. Additional details regarding an implantable medical device with long-range wireless capabilities can be found in U.S. patent application Ser. No. ______, filed on Dec. 23, 2002, and entitled “Method and Apparatus for Enabling Data Communication Between An Implantable Medical Device and A Patient Management System”which is assigned to the assignee of the instant patent application and expressly incorporated herein by reference.

According to another embodiment, the device 202 is equipped with short-range wireless capabilities for communicating with a repeater 506 via a short range communications link 522. The repeater 506 may then relay signals between the device 202 and the patient management system through a wired connection 518. Alternatively, the repeater 506 may communicate with the patient management system 200 through a long-range wireless communications link 526 established with the wireless communications system 512.

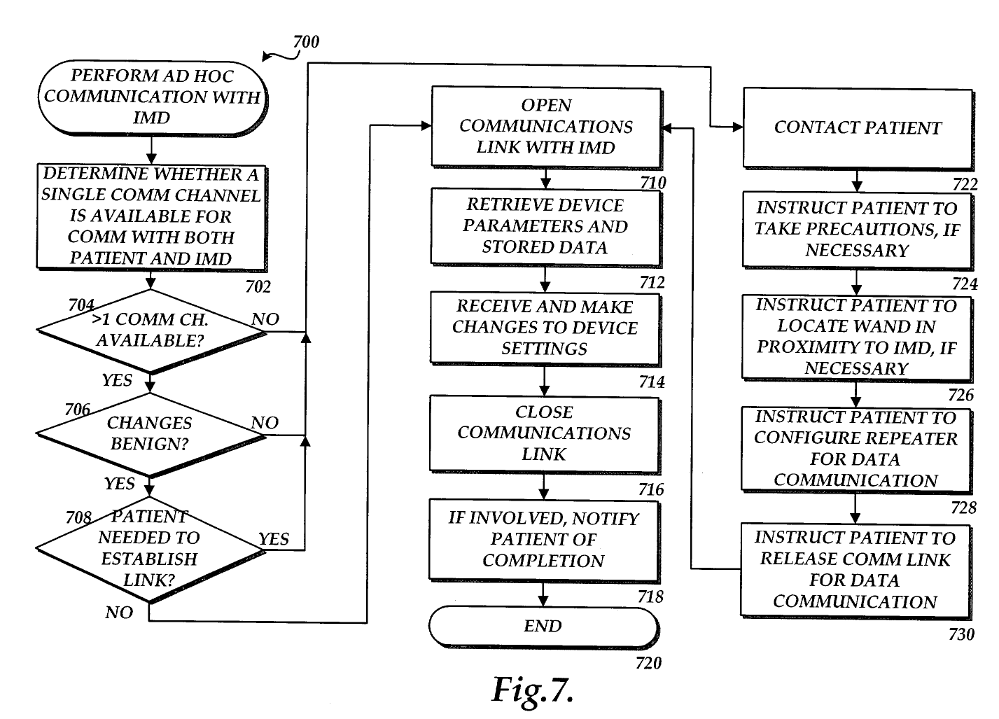

Turning now to FIG. 7, an illustrative routine 700 for performing ad hoc communication with an IMD will be described. As described above, the communications session takes place between the patient management system 200 and the device 202. During the communications session, device parameters may be adjusted, patient data stored in the device may be retrieved, and other actions may be performed. For instance, the physician or clinician may access data stored in the device 202, such as an electrogram, and can use this information to make device adjustments while monitoring the patient's condition in real time. In determining the number of communications channels, the ITU can facilitate both data and voice transfer over a single physical link, but it's use is dependent on the type of equipment available to the clinician. For example, a computer with multi-media capability could be used to multiplex data and voice communications with a suitably configured ITU over a single phone line connection.

The routine 700 begins at block 702, where a determination is made as to whether only a single communications channel exists for communication with both the patient 502 and the device 202. A single communications channel may exist, for example, when the repeater 506 utilizes a telephone line for communication with the patient management system 200 and the telephone line is also used by the patient 502 for voice communication. Data regarding the type and number of communications channels available to the patient 502 may be stored at the patient management system 200.

At block 704, the routine 700 branches to block 722 if only one communications channel is available for communicating with both the device 202 and the patient 502. If more than one communications channel exists for communication with both the device 202 and the patient 502, the routine 700 continues from block 704 to block 706. This occurs, for instance, if the patient 502 maintains one telephone line for voice communication and a separate telephone line for use by the repeater. Alternatively, this may occur if the repeater utilizes a wireless communications link or a dedicated network communications link for communicating with the patient management system 200.

At block 706, a determination is made as to whether the communications session will involve changes to the device 202 that may effect the health or well being of the patient 502. If no changes are to be made to the device 202 that will effect the health or well being of the patient 502, simply retrieving data from the device 202 for instance, the patient 502 does not necessarily need to be notified that a communications session is taking place. If changes are to be made to the device 202 that may effect the health or well being of the patient 502, the patient should be notified prior to any communication with the device 202. Accordingly, if the changes to the device 202 are not benign, the routine 700 branches to block 722. If the changes are benign, the routine 700 continues to block 708.

At block 708, a determination is made as to whether the participation of the patient 502 is necessary to establish a communications link with the device 202. This may be necessary, for instance, if the repeater 506 utilizes a wand 504 for communication with the device 202 as shown in FIG. 5. This may also be necessary if the repeater 506 must be manually placed into a communications mode prior to the initiation of a communications session. The participation of the patient 502 may also be required to perform other tasks necessary to establishing a communications link between the patient management system 200 and the device 202. If the participation of the patient 502 is necessary to establish a communications link, the routine 700 branches to block 722. If the participation of the patient 502 is not necessary, the routine 700 continues to block 710.

At block 722, the patient 502 is contacted. Typically, a telephone call is made to a telephone in proximity to the patient 502 to communicate directly with the patient 502. In particular, an automated voice response system maintained as a part of the patient management system 200 communicates with the patient 502 answering the phone or, alternatively, a clinician or physician may speak directly with the patient 502. From block 722, the routine 700 continues to block 724, where the clinician or the automated voice response system explains the adjustments to be made to the patient's device 202 and requests any special precautions to be taken by patient. For instance, the patient may be required to lie down while the communication session is in progress.

From block 724, the routine 700 continues to block 726, where the patient is instructed to place the wand 504 in proximity to the device 202 if necessary. As described above, in some embodiments of the invention, the patient places a wand 504 or pager device over his IMD 202 and the wand 504 or pager device is either wired to a repeater or to the patient management system or the wand or pager is wirelessly connected to the repeater or the remote management site. These embodiments may require that the patient hold the wand or pager in place. Thus, these embodiments may not be acceptable in all conditions such as where the patient may lose consciousness and device communications may be lost.

From block 726, the routine 700 continues to block 728, where the patient is instructed to configure the repeater 506 for data communication with the patient management system 200. For instance, if only a single telephone line is available, the patient 502 may be required to configure the repeater 506 to answer the next incoming telephone call. In this manner, the patient management system 200 can establish a data communications link with the repeater 506 on a subsequent call to the repeater. Once the patient 502 has performed this task, the patient 502 may be instructed at block 730 to release the telephone line for communication with the repeater 506 and the device 202. It should be appreciated that in the embodiments of the invention wherein the device 202 has long range wireless capabilities, the patient need not be instructed to utilize a wand 504, to configure a repeater 506, or to release a communications line. The patient may, however, be instructed to take precautions prior to establishing a communications session, if necessary.

From blocks 704, 706, 708, and 730, the routine 700 continues to block 710. At block 710, the clinician uses the patient management system 200 to begin communicating with the device 202. As described above, a connection may be made through a wireless communications system to a device 202 or repeater 506 equipped with wireless functionality or a wireline based communications link may be established with a repeater device configured for communication over a wired link. The repeater 506 or device 202 may then communicate patient information to the patient management system 200. For instance, at block 712, device parameters and data stored in the device 202 are retrieved. The physician or clinician can then view real-time patient information and make adjustments to parameters or settings of the IMD 202 in real-time. Typically, the physician or clinician can interactively tune parameters of the IMD 202. This process is performed at block 714.

Once the communications session between the device 202 and the patient management system 200 has been completed, the communications link is closed at block 716. The routine 700 then continues to block 718, where the patient is notified that the session has completed and instructed to resume their normal activities. If the patient was not notified prior to establishment of the communications session, the patient will not be notified following the completion of the communications session. From block 718, the routine 700 continues to block 720, where it ends.

Based upon the foregoing, it should be appreciated that the present invention provides methods and systems for ad hoc real-time communication with an implantable medical device. It should be understood that using embodiments of the present invention, no trip is necessary to a physician's clinic or to a clinician. Thus, it should be understood that the invention spatially decouples a patient and the physician or clinician who wants to download data from the patient's IMD or to make adjustments to the IMD. Although the invention has been described in language specific to computer structural features, methodological acts and by computer readable media, it is to be understood that the invention defined in the appended claims is not necessarily limited to the specific structures, acts or media described. Therefore, the specific structural features, acts and mediums are disclosed as exemplary embodiments implementing the claimed invention. Since many embodiments of the invention can be made without departing from the spirit and scope of the invention, the invention resides in the claims hereinafter appended.

We claim:

1. A method for communicating with an implantable medical device implanted within a patient, wherein the implantable medical device is operative to communicate with a patient management system, the method comprising:
determining whether the patient must be notified prior to communicating with the implantable medical device;
in response to determining that the patient must be notified prior to communicating with the implantable medical device, communicating to the patient one or more actions to be taken prior to initiating communication with the implantable medical device;
in response to the completion of the actions, using the patient management system to initiate a communications link with the implantable medical device; and
communicating with the implantable medical device via the communications link.

2. The method of claim 1, wherein determining whether the patient must be notified prior to communicating with the implantable medical device comprises determining whether only a single communications channel exists for communication with both the patient and the implantable medical device.

3. The method of claim 1, wherein determining whether the patient must be notified prior to communicating with the implantable medical device comprises determining whether changes may be made to the implantable medical device during communication with the implantable medical device that may effect the health or well being of the patient.

4. The method of claim 1, wherein determining whether the patient must be notified prior to communicating with the implantable medical device comprises determining whether the participation of the patient is necessary to establish a communications link with the implantable medical device.

5. The method of claim 1, wherein communicating to the patient one or more actions to be take prior to initiating communication with the implantable medical device comprises communicating to the patient an instruction to take special precautions necessary.

6. The method of claim 1, wherein communicating to the patient one or more actions to be take prior to initiating communication with the implantable medical device comprises communicating to the patient an instruction to locate their the implantable medical device proximate to a repeater device necessary to establish communication between the implantable medical device and the patient management system.

7. The method of claim 6, wherein communicating to the patient one or more actions to be take prior to initiating communication with the implantable medical device comprises instructing the patient to configure the repeater device for communication with the patient management system.

8. The method of claim 1, wherein communicating with the implantable medical device via the patient management system comprises retrieving one or more device parameters from the implantable medical device.

9. The method of claim 8, wherein communicating with the implantable medical device via the patient management system further comprises retrieving data stored in the implantable medical device regarding the health of the patient.

10. The method of claim 9, wherein communicating with the implantable medical device via the patient management system further comprises changing the device parameters stored in the implantable medical device.

11. The method of claim 10, further comprising notifying the patient of the completion of the communication with the implantable medical device.

12. A computer-readable medium encoded with computer-readable instructions which, when executed by a computer, will cause the computer to perform the method of claim 1.

13. A computer-controlled apparatus capable of performing the method of claim 1.
